(19) United States
(12) Patent Application Publication
(10) Pub. No.: US 2025/0302757 A1
(43) Pub. Date: Oct. 2, 2025

(54) Lipid Microcapsules for Viable and Stable Probiotics

(71) Applicant: Lonza Greenwood LLC, Greenwood, SC (US)

(72) Inventors: Jowell Bolivar, Greenwood, SC (US); Tyler White, Greenwood, SC (US); Shane Durkee, Morristown, NJ (US); Stanislaw Glab, Greenwood, SC (US); Kalee Aiken, Greenwood, SC (US)

(21) Appl. No.: 18/861,643

(22) PCT Filed: May 25, 2023
(86) PCT No.: PCT/US2023/023504

§ 371 Date: Oct. 30, 2024

Related U.S. Application Data
(60) Provisional application No. 63/345,975, filed on May 26, 2022.

(51) Int. Cl. A61K 9/50; A61K 9/19; A61K 9/48; A61K 35/741

(52) U.S. Cl. CPC A61K 9/5015; A61K 9/19; A61K 9/4858; A61K 9/4866; A61K 35/741

(57) ABSTRACT

The present disclosure is directed to a health composition that includes lipid multiparticulates containing one or more probiotics in a lipid matrix formed at least in part from excipients and/or encapsulants having a melting point of less than about 70° C. The one or more probiotics maintain a high level of viability after incorporation into the lipid matrix. In addition, the lipid multiparticulates can maintain the viability and stability if the at least one probiotic in high temperature and high humidity environments.

### BACKGROUND

Probiotic cell matter includes probiotic organisms, such as viable bacteria and yeast cells. Probiotic cell matter used as a supplement has rapidly gained in popularity due to the health benefits obtained when consumed or administered to a mammal. Probiotic cell matter, for instance, can improve immune system reaction, reduce inflammation, and/or reduce gastrointestinal discomfort in mammals, and can even increases the presence of T cell types in the body and/or increases the production of cytokines in the body. Further, probiotic cell matter, has also been shown to modulate immune system responses in mammals either directly or indirectly. such as by maintaining or repairing epithelial barriers or by increasing the production of fatty acids, such as short chain fatty acids that have anti-inflammatory properties, through a number of distinct mechanisms.

Probiotic cell matter, or probiotics, derive many of their benefits from the active or viable cells being ingested by the mammal. However, forming supplements that contain viable probiotics has proven to be a challenge, as probiotics are easily denatured during the processing required to form supplements, such as high temperature and pressure, and are not compatible with high water activity or high moisture environments. This makes formulating supplements containing viable probiotics for mammal consumption problematic, as many active ingredients, also referred to as nutraceutical ingredients, such as vitamins, minerals, botanical extracts, enzymes, omega-3, fiber sources, prebiotics, and postbiotics have high water-activity or antimicrobial properties that can kill or denature probiotics. As would be understood, these instabilities makes probiotics unsuitable for incorporation into many delivery methods, such as tablets, ready-to-mix beverages, capsules, and the like, for the problems stated above in regards to processing, additional active ingredients, and stability during storage.

In addition, it is generally desirable for the probiotic to maintain viability through the gastrointestinal tract in order to be delivered to the intestines and colon to provide optional benefit. However, probiotics are not resistant to low pH conditions in the stomach, resulting in low viability after passing through the stomach.

Attempts to provide probiotics having improved viability after processing and ingestion have been proposed, such as by coating or encapsulating the probiotic. However, existing methods and products require heat above 60° C. or the inclusion of water or moisture in the process, resulting in low probiotic viability in the final product. (e.g. a low percentage of viable probiotics after processing or storage, as compared to the initial viability). In addition, such processes utilize high melting point coatings that are incompatible with many supplement products, such as ready-to-mix beverages, as the constituents of the coating have melting points above the desired administration temperature of the product, and where clear liquid appearances are expected. Moreover, such methods are further detrimental to certain species of probiotic's that are highly sensitive to temperature and water content, such as Lactobacilli and Bifidobacteria species, which often exhibit probiotic loss under existing processing methods.

Therefore, it would be a benefit to provide a probiotic composition that overcomes one or more of the above deficiencies. In one aspect, it would be beneficial to provide a probiotic composition that exhibits high recovery of viable probiotics after formation of the probiotic composition. It would be another benefit to provide a probiotic composition that is stable in the presence of one or more active nutraceutical ingredients, including active ingredients that have a high moisture content, high water-activity, or antimicrobial properties. In addition, it would be another benefit to provide a probiotic composition that is resistant to high acid conditions, such as stomach pH.

### SUMMARY

The present disclosure is generally directed to a probiotic composition that includes lipid multiparticulate particles and at least one probiotic. The lipid multiparticulate particles include a lipid matrix, where the at least one probiotic, alone or in combination with a paraprobiotic, is dispersed within the lipid matrix. In addition, at least a portion of the lipid matrix is formed from one or more encapsulants, one or more excipients, or a combination thereof, having a melting point of about 70° C. or less, and greater than about 80% of the probiotics contained in the lipid matrix are viable probiotics.

The present disclosure is also generally directed to a health composition that includes at least one nutraceutical ingredient and lipid multiparticulate particles. The at least one nutraceutical ingredient has a water-activity of greater than 0.2, is an antimicrobial ingredient, or a combination thereof and the lipid multiparticulate particles include a lipid matrix, and wherein at least one probiotic, alone or in combination with a paraprobiotic, is dispersed within the lipid matrix. In addition, at least a portion of the lipid matrix is formed from one or more encapsulants, one or more excipients, or a combination thereof, having a melting point of about 70° C. or less.

In one aspect, the lipid matrix is formed from one or more encapsulants, one or more excipients, or a combination thereof, having an average melting point of less than about 70° C. In a further aspect, at least a portion of the one or more encapsulants, one or more excipients, or a combination thereof, have a melting point of less than about 50° C., preferably less than about 40° C. Moreover, in an aspect, the lipid matrix further includes at least one mild flow point excipient, high flow point excipient, or a combination thereof, the at least one mild flow point excipient, high flow point excipient, or a combination thereof having an average melting point of less than about 65° C. In another aspect, the lipid multiparticulate particles have an average particle size of greater than 1 μm, generally greater than 10 μm, typically from about 40 microns to about 3000 microns, such as from about 100 microns to about 2000 microns, such as from about 150 microns to about 1500 microns.

Additionally or alternatively, in an aspect, the composition includes a nutraceutical ingredient, where the at least one nutraceutical ingredient includes an herbal extract, a botanical extract, a coloring, a flavoring, an enzyme, a vitamin, a mineral, an omega-3 fatty acid, a prebiotic, a postbiotic, a fiber source, or a combination thereof.

In yet another aspect, greater than about 90% of the probiotics contained in the lipid matrix are viable probiotics, preferably wherein the percent recovery is after exposure to processing and/or a temperature of 55° C. for at least about 2 hours, such as about 6 hours, preferably up to about 24 hours. Furthermore, in one aspect, the probiotic is present in the lipid multiparticulate particles in an amount from about 1% to about 80% by weight, such as in an amount from about 10% to about 75% by weight, more particularly in an amount from about 25% to about 70% by weight based on a total weight of the lipid multiparticulate particles. In another aspect, least a portion of the at least one probiotic is a freeze dried probiotic powder.

Furthermore, in an aspect, the composition is in a form of a capsule or a powder or suspended in a liquid, or wherein the composition is or is contained in, a ready-to-mix beverage, oatmeal, cereal, a nutritional bar, pet chews, pet treats, or a combination thereof, and/or wherein the composition is packaged in a resin material, such as a high density polyethylene, aluminum, or a desiccant-lined vial.

In one aspect, the lipid matrix includes a fatty alcohol, a fatty acid, a fatty acid ester of a glycol and a poly glycol, a fatty acid ester of glycerol, polyglycerol, a polyglycolized glyceride, a C10-C18 triglyceridesstearoyl polyoxylglyceride, a lauroyl macrogol-32 glyceride, a caprylocaproyl macrogol-8 glyceride, an oleoyl macrogol-6 glyceride, a linoleoyl macrogol-6 glyceride, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, a propylene glycol fatty acid ester, esterified alpha-tocopheryl polyethylene glycol succinate, a propylene glycol monolaurate (C12) ester, polyoxyl 35 castor oil, polyoxyl 40 hydrogenated castor oil, a lecithin, vitamin E, tocopheryl polyethylene glycol succinate (TPGS), a sugar fatty acid ester, a sorbitan fatty acid ester, a polyoxyethylene sorbitan fatty acid ester, a polyoxyethylene-polyoxypropylene copolymer, rosemary extract, propylene glycol, triacetin, isopropyl myristate, diethylene glycol monoethyl ether, polyethylene glycol, glycerol, mixtures or combinations thereof. Additionally or alternatively, in an aspect, the lipid matrix includes a wax, a fatty alcohol, and a fatty acid, preferably where the wax comprises candelilla wax, where the fatty alcohol comprises stearyl alcohol, and where the fatty acid comprises stearic acid. Additionally or alternatively, in an aspect, the lipid matrix further contains a surfactant, a cross-linked carboxymethyl cellulose salt, or combinations thereof. Moreover, in one aspect, the surfactant includes a polysorbate, a laureth sulfate, or mixtures thereof. Furthermore, in yet another aspect, the composition can further include one or more flow aids, antioxidant, dispersing agent and/or a flavoring or sweetener, preferably where the one or more flow aids, antioxidant, dispersing agent and/or a flavoring or sweetener are not dispersed in the lipid matrix.

The present disclosure is also generally directed to administering a composition according to any one or more of the above aspects to a mammal, where the method includes orally administering to a mammal any aspects of the composition discussed above, where each dosage administered to the mammal contains the at least one probiotic in an amount from about 0.5 B CFU to about 150 B CFU, such as about 1 Billion (B) Colony Forming Units (CFU) to about 75 B CFU and more particularly between about 2.5 B CFU to about 30 B CFU. In one aspect, the composition is formulated such that the probiotic is released from the composition over a period up to about 30 hours after oral administration by a mammal, such as in period of time from about 0.5 hours to 24 hours after administration by a mammal, and more particularly in a period of time from about 1 hour to about 20 hours after administration by a mammal.

In addition, the present disclosure is also generally directed to a method of increasing bioavailability of at least one probiotic in a mammal. The method includes forming a composition according to any one or more of the above aspects, and administering the composition to a mammal.

Other features and aspects of the present disclosure are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 is an illustration of lipid microcapsules of Lactobacillus plantarum TWK10® formed according to the present disclosure;

FIG. 2 is an illustration of an example of forming lipid microcapsules according to the present disclosure, and the process for recovering probiotics from the lipid microcapsules for determining viability;

FIG. 3 is a comparison of using traditional plate enumeration as compared to flow cytometry as discussed in Example 3 to determine viability in recovered probiotics;

FIG. 4 is a chart showing viable probiotics recovered from lipid microcapsules formed according to Example 3 after mixing for 6 hours at processing temperatures of 55-58° C. using flow cytometry;

FIG. 5 is a chart showing viable probiotics recovered from lipid microcapsules formed according to Example 3 after mixing for 24 hours at processing temperatures of 55-58° C. using flow cytometry;

FIG. 6 is a chart showing viable probiotics recovered from lipid microcapsules formed according to Example 3 after storage for six months at a temperature of 25° C. and 60% relative humidity using flow cytometry; and

FIG. 7 is a chart showing viable probiotics recovered from lipid microcapsules formed according to Example 3 after storage for six months at a temperature of 40° C. and 75% relative humidity using flow cytometry.

Repeat use of reference characters in the present specification and drawings is intended to represent same or analogous features or elements of the invention.

### Definitions

As used in this application and in the claims, the singular forms “a,” “an,” and “the” include the plural forms unless the context clearly dictates otherwise. Additionally, the term “includes” means “comprises.” The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

Unless otherwise indicated, all numbers expressing quantities of ingredients, properties such as molecular weight, percentages, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited. As used herein, the terms “about,” “approximately,” or “generally,” when used to modify a value, indicates that the value can be raised or lowered by 10%, such as, such as 7.5%, 5%, such as 4%, such as 3%, such as 2%, such as 1%, and remain within the disclosed aspect. Moreover, the term “substantially free of” when used to describe the amount of substance in a material is not to be limited to entirely or completely free of and may correspond to a lack of any appreciable or detectable amount of the recited substance in the material. Thus, e.g., a material is “substantially free of” a substance when the amount of the substance in the material is less than the precision of an industry-accepted instrument or test for measuring the amount of the substance in the material. In certain example embodiments, a material may be “substantially free of” a substance when the amount of the substance in the material is less than 10%, less than 9%, less than 8%, less than 7%, less than 6%, less than 5%, less than 4%, less than 3%, less than 2%, less than 1%, less than 0.5%, or less than 0.1% by weight of the material.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” means by weight as a percentage of the total weight or relative to another component in the composition.

The phrase “effective amount” means an amount of a compound that promotes, improves, stimulates, or encourages a response to the particular condition or disorder or the particular symptom of the condition or disorder.

The term “therapeutically effective amount” as used herein, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “nutraceutical” and refers to any compound added to a dietary source (e.g., a food, beverage, or a dietary supplement) that provides health and/or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved joint health, resilience, and recovery, and can include without limitation human, canine, equine, feline, bovine, ovine, or porcine mammals. For purposes of this application. “mammal” does include human subjects.

The term “supplement” means a product in addition to the normal diet but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

As used herein, “healthy” refers to the absence of illness or injury.

As used herein, the term “flow point” is the temperature at which any portion of the mixture becomes sufficiently fluid that the mixture, as a whole, may be atomized. Generally, a mixture is sufficiently fluid for atomization when the viscosity of the molten mixture is less than 20,000 cp, or less than 15,000 cp, or less than 10,000 cp, less than 5000 cp, or even less than 1000 cp. The viscosity can be measured by a controlled stress rheometer, which measures viscosity as a function of temperature, and may use either a shear-type or rotational rheometer. As used herein, melting point refers to the temperature that marks the midpoint of the transition from a solid crystalline or semi-crystalline state to a liquid state. As measured by DSC and other melting point detection apparatuses, the melting point is the temperature where upon heating the solid material, the maximum exothermic heat flow occurs. In general, melting point will be used in reference to relative pure single component materials such as some actives or essentially single component excipients (e.g. stearyl alcohol) and flow point will be used in reference to multi-component materials or mixtures.

As used herein, the term “semi-solid” is a solid at ambient temperature (23° C.) but becomes a liquid at temperatures above 30° C. or 40° C., or at body temperature.

Unless otherwise indicated, “capsule” means a container suitable for enclosing solids or liquids and includes empty capsule shells and components thereof such as caps and bodies that may be assembled together to form the capsule.

As used herein, by “active” or “active ingredient” is meant a drug, medicament, pharmaceutical, therapeutic agent, nutraceutical, or other compound that may be desired to be administered to the body. The active ingredient may be a “small molecule,” generally having a molecular weight of 2000 Daltons or less. The active ingredient may also be a “biological active.” Biological active ingredients include proteins, antibodies, antibody fragments, peptides, oligonucleotides, vaccines, and various derivatives of such materials. In one embodiment, the active ingredient is a small molecule. In another embodiment, the active ingredient is a biological active. In still another embodiment, the active ingredient is a mixture of a small molecule and a biological active. Also as used herein, the terms “active ingredient”, “first active ingredient”, “second active ingredient”, etc. may be used to denote active ingredients located in different places within the particle, such as those located in the core or those located in the one or more outer layers. However, the terms “first” or “second” do not necessarily denote that the first active ingredient is different from the second active ingredient. For example, in certain embodiments, the active ingredient contained within the core may be the same as the second active ingredient contained within an outer layer disposed on the core. While in certain other embodiments, the active ingredient contained within the core may be different from the second active ingredient contained within an outer layer disposed on the core

Unless otherwise indicated, “dosage form” refers to a solid composition comprising an active ingredient.

As used herein, the term “particle” refers a portion or quantity of material(s), such as a small portion or quantity of material(s). For example, as provided herein, the term particle may refer generally to a composition containing a core and one or more outer layers surrounding the core. In some embodiments, the particle(s) described may be generally spherical in shape. The term “particle” as used herein includes or may be used interchangeably with the following: pellet, beadlet, multiparticulates, particulates, spheres, including microspheres, seeds, and the like. The term particle as used herein is not limited to only a particle formed by certain methods or processes. Indeed, the particle(s) described herein may be formed by any suitable process. Certain suitable processes include, but are not limited to, melt spray congealing, spheronization, extrusion, compression, powder layering, liquid layering, pelletization by melt and wet granulation, and combinations thereof. The particle(s) as described herein may be solid or semi-solid particles. In some embodiments, the particles describe herein can include both solid and semi-solid compositions contained on or within the particle itself.

“Probiotic cell mater” or “Probiotic” as used herein refers to one or more probiotic organisms, including viable bacterial and yeast cells, as well as paraprobiotics, which include the killed or inactivated cells of probiotic organisms and/or the crude cell fractions of probiotic organisms including probiotic derivatives, which include the processed cell components of probiotic organisms. However, as will be discussed in greater detail below, “percent recovery yield” of viable probiotics are based upon the amount of viable probiotic contained in the probiotic cell matter or probiotic prior to processing. Therefore, “percent recovery yield” is a percentage of the initial, pre-processing, viable probiotic recovered after processing or stability testing.

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

The present disclosure is generally directed to lipid multiparticulates (also referred to as lipid multiparticulate particles) containing one or more probiotics and/or probiotic cell matter dispersed in a lipid matrix that is formed at least in part from excipients and/or encapsulants having a low melting point (which may also be referred to as a low flow point, as discussed in greater detail below), alone or in combination with having a low water activity. Namely, the present disclosure has surprisingly found that the use of such low melting point excipients and/or encapsulants allows a composition to be formed that overcomes many of the deficiencies of existing probiotic supplements. Namely, by forming at least a portion of the lipid matrix with an excipient and/or encapsulant having a low melting point, alone or in combination with having a low water activity, a unique and gentle encapsulation process can be used to form the lipid multiparticulate, greatly increasing the viability and stability of the probiotic in the composition.

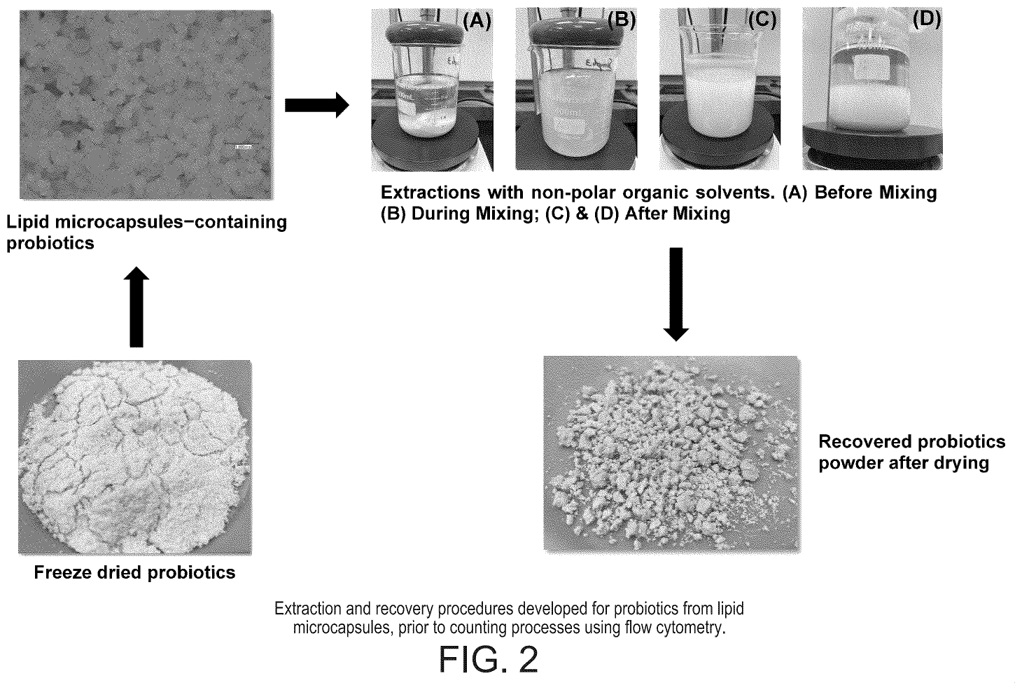

For instance, in one aspect, one or more probiotics that were dispersed in a lipid matrix may be recovered and tested to determine the percentage of the probiotics that remain viable after processing and incorporation into the lipid multiparticulate, which will be discussed in greater detail below in regards to FIG. 2. Namely, the present disclosure has unexpectedly found that about 80% or more of the viable probiotics dispersed in the lipid matrix remain viable after formation of the lipid multiparticulate based upon the initial amount of viable probiotics incorporated into the lipid multiparticulate, such as about 82.5% or more, such as about 85% or more, such as about 87.5% or more, such as about 90% or more, such as about 92.5% or more, such as about 95% or more of the viable probiotics utilized to form the lipid multiparticulate remain viable, or any ranges or values therebetween.

Furthermore, it was surprisingly found that the above percentages of recovered viable probiotics are observed even after processing at temperatures of about 40° C. or more, such as about 45° C. or more, such as about 50° C. or more, such as about 55° C. or more such as about 57.5° C. or more, up to about 60° C. or less, or any ranges or values therebetween, even when processed at such temperatures for about 2 hours or more, such as about 6 hours or more, such as up to about 24 hours.

Moreover, the present disclosure has also unexpectedly found that the probiotic containing lipid multiparticulates exhibited the above percentages of recovered viable probiotic even after storage for about 2 weeks or more, such as about 3 weeks or more, such as about 4 weeks or more, such as about 6 weeks or more, such as about 8 weeks or more, such as about 10 weeks or more such as up to about 24 weeks or more, in a sealed container, which can include a desiccant vial, such as a CSP™ Vial, a polymeric sachet, such as one formed from high density polyethylene (HDPE) for example, an aluminum sachet, or other sealed packaging as known in the art. In addition, the improved stability and recovery were observed even at temperatures of about 25° C. or greater, such as about 30° C. or greater, such as about 35° C., or greater, even up to about 40° C., or any ranges or values therebetween. In addition, it was surprisingly found that the above percentages of recovered viable probiotics was observed when exposed to high relative humidity's of about 50% or greater, such as bout 60% or greater, such as about 65% or greater, such as about 70% or greater, such as about 75% or greater, or any ranges therebetween. Thus, it was unexpectedly found that the high percentage of viable probiotics were observed even at 25° C. and 60% relative humidity, or even at highly stressful conditions for the probiotics at 40° C. and 75% relative humidity. It should also be understood, as shown in greater detail in the examples, that viability was observed even at combinations of time, heat, and humidity, and thus, that viability was observed even when two or more of the above environments were experienced together (e.g. temperature+relative humidity and/or time). Nonetheless, in one aspect, it was found that percent viability can be further exhibited with a unique combination of the lipid multiparticulates and a desiccant lined vial, particularly in conditions not normally considered stable for probiotics in any packaging, such as high temperatures and/or humidity, which is illustrated in greater detail in the examples below. Furthermore, it should be understood that percent viability in recovered probiotics may be measured using flow cytometry or a standard plate method, as will be discussed in greater detail in regards to FIG. 2 below.

Furthermore, in one aspect, the lipid multiparticulate particles may be placed into a capsule, formed into a tablet, placed in a soft-gel, placed in a gummy, may be alternatively ingested directly by a mammal as a powder or can be incorporated into a beverage or other food item, as will be discussed in greater detail below. Namely, as briefly discussed above, a further unexpected benefit of using at least one low melting point excipient and/or encapsulant in the lipid matrix is that it improves the compatibility of the lipid multiparticulate with various food and beverage products. For instance, due to the lower melting point of the lipid multiparticulate, the lipid multiparticulate may be used to protect the one or more probiotics until the supplement containing the probiotic (or the lipid multiparticulate containing probiotics alone) are incorporated into a drink as a ready-to-mix beverage or added into or onto any other food or beverage, or added onto or into pet food and/or pet treats, at which point the lipid matrix may dissolve, yielding an stable probiotic with a neutral taste. Thus, lipid multiparticulates according to the present disclosure may be used with lower temperature food and beverages due to the low melting point of the constituents of the lipid matrix. However, as will be discussed in greater detail, the lipid multiparticulate can also be formulated to maintain the particulate prior to ingestion, so as to stabilize the probiotic into the intestine and/or colon of a mammal, and may therefore be formulated so as to remain in particulate form when added to foods or beverages.

For instance, in one aspect, the lipid multiparticulate particles include a lipid matrix that, in one aspect, can stabilize the probiotic when the particles are in contact with an environment that would otherwise inactivate the probiotic, prior to delivery to the desired portion of the digestive system of the mammal (e.g. the small intestine and/or colon). Namely, in one aspect, the lipid multiparticulate may protect and/or stabilize the one or more probiotics until an environment is encountered which cause the probiotic to be released from the lipid multiparticulates, such as in the digestive systems of a mammal that has been orally administered or otherwise ingested the lipid multiparticulates, protecting the probiotic in a viable state through the stomach of a mammal.

In addition, due to the gentle nature of the process achievable due at least in part to the lipid matrix formed from one or more low flow point excipients and/or encapsulants, the present disclosure has also surprisingly found that lipid multiparticulates can be formed using freeze dried probiotics, and still achieve high viability according to the above ranges in recovered probiotics. Namely, freeze dried probiotics are highly susceptible to viability loss due to fracture of cell membranes. However, the process of the present disclosure has been found to disperse or encapsulate the freeze dried probiotic without causing further harm or degradation to the freeze-dried probiotic, and instead provides a coating that maintains a high level of viability in recovered probiotics, such as the ranges set forth above.

Also, in one aspect, the present disclosure has surprisingly found that the lipid multiparticulates stabilize non-spore forming probiotics. Such stabilization was found to increase shelf-life and/or make non-spore forming probiotics amenable to food and beverage applications by way of providing stability against temperature and/or pH challenges. Unexpectedly, non-spore forming probiotics in the lipid multiparticulates maintained viability when exposed to various environmental challenges, such as, for example passage of time, heat, humidity, pH, or a combination thereof. For instance, non-spore forming probiotics in the lipid multiparticulates maintained viability after processing at temperatures of about 40° C. or more, after storage at temperatures of about 25° C. or more and/or exposure to about 60% or more humidity, after passing through the low pH conditions in the stomach, and/or for a period up to about 30 hours after oral administration.

Furthermore, the present disclosure has also unexpectedly found that by suspending one or more probiotics in a lipid matrix of a lipid multiparticulate, the lipid multiparticulate may be combined with additional nutraceutical ingredients to form a supplement, without degrading or causing viability loss to the probiotic. Namely, as noted above, the lipid multiparticulate can provide stability and protection to the probiotic, even when processed at low temperatures/when the lipid matrix includes one or more low melting point excipients or encapsulants. Thus, the suspended probiotic can be used in combination with nutraceutical ingredients that would have denatured the probiotic in previous attempts to form a supplement including a probiotic and one or more nutraceutical ingredients, such as nutraceutical ingredients having a water activity of greater than 0.2, antimicrobial ingredients, and the like. Thus, in one aspect, a health composition or supplement containing the probiotic containing lipid multiparticulate can include one or more additional nutraceutical ingredients, such as vitamins, minerals, botanical extracts, herbal extracts, enzymes, omega-3 fatty acids, fiber sources, prebiotics, postbiotics, flavorings, colorings, or combinations thereof.

Nonetheless, as discussed above, the lipid multiparticulate contains one or more probiotics, at least a portion of which are considered to be viable probiotics. However, in one aspect it should be understood that substantially all of the probiotics incorporated in to the lipid multiparticulate are viable.

Probiotic organisms that may be used in the accordance with the present disclosure to provide probiotics or probiotic cell matter include various beneficial bacteria and yeast. For instance, a probiotic organism may comprise gram-positive bacteria, gram-negative bacteria, aerotolerant bacteria, anaerobic bacteria, microaerophilic bacteria, non-spore-forming bacteria, or various different eukaryotic cells, such as yeast.

In one aspect, for instance, the probiotic organism used in accordance with the present disclosure comprises a type of bacteria. The bacteria may be selected from various different phyla including strains selected from the Firmicutes, the Gracilicutes, or the Mendocutes, and would include Bacteroidetes, Actinobacteria, Proteobacteria, Lactobacteria, the Bacilli, Verrucomicrobia, Faecalibacteria, Thermophiles, Lactobacillus, Bifidobacterium, and the Clostridias. Specific examples of probiotics that may be used include, but are not limited to, Escherichia coli, Bacteroides fragilis, various Bifidobacteria and Lactobacteria, including Lactobacteria casei, Bifidobacterium longum ssp infantis, Lactobacteria johnsonii, Lactobacteria rhamnosus, Lactobacteria reuteri, Lactobacteria acidophilus, Lactobacteria paracasei, Lactobacillus plantarum, and Lactobacillus lundensis, Bacillus coagulans, Bacillus subtilis, Faecalibacteria prausnitzil, Enterococcus faecium, Streptococcus salivarius, Clostridia butyricum, Akkermansia muciniphila, or mixtures thereof. In one aspect, for instance, the probiotic organism used in accordance with the present disclosure comprises bacteria of the genus Lactobacillus. Specific examples of probiotics that may be used include, but are not limited to, L. acidophilus, L. acidophilus La-14®, L. rhamnosus HN001, L. rhamnosus LGG®, L. casei, L. plantarum, L. brevis, L. fermentum, or mixtures thereof.

In another aspect, for instance, the probiotic organism used in accordance with the present disclosure comprises bacteria of the genus Bifidobacterium. Specific examples of probiotics that may be used include, but are not limited to, B. lactis HN019, B. lactis BL04®, B. animalis subsp. lactis BB-12®, B. bifidum, B. breve, B. longum, or mixtures thereof.

In an alternative aspect, eukaryotic cells may be used as the probiotic organism. For instance, the probiotic organism may comprise Saccharomyces cerevisiae.

In another aspect, the probiotic organism can include bacteria or yeast cells that have been treated or altered but remain viable. For instance, probiotic organism cells that have been lyophilized, or freeze-dried, but can be reconstituted, or probiotic organism cells that have undergone heat treatment, but retain viability, may be used in accordance with the present disclosure.

Nonetheless, in one aspect, at least one probiotic is from a genus of Lactobacilli, Bifidobacteria, or a combination thereof, or other probiotics that provide a benefit to a small intestine or a colon of a mammal.

Paraprobiotics that may be used in accordance with the present disclosure include the killed or inactivated cells of probiotic organisms and the cell fractions of probiotic organisms. However, as discussed above, it should be understood that at least a portion of the probiotics incorporated into the lipid multiparticulate are viable probiotics. Thus, in one aspect, paraprobiotics can be used in conjunction with viable probiotics, and, in one aspect, the probiotic may not contain substantially any paraprobiotics/inactivated cells prior to incorporation into the lipid matrix. Nonetheless, in one aspect, the paraprobiotic may comprise bacteria or yeast cells that have undergone heat treatment and are no longer viable but are still able to provide health benefits when consumed or administered to a mammal. Alternatively, the paraprobiotic may comprise viable or nonviable cells possibly subjected to changes in pH, increased pressure, milling, or the like, and in various combinations.

In yet another aspect, the paraprobiotic may comprise crude cell fractions containing, for instance, metabolites or hydrolysates. For example, protein hydrolysates extracted from yeast may be used in accordance with the present disclosure.

Other paraprobiotics that may be used in accordance with the present disclosure include probiotic derivatives that may include processed cell components of probiotic organisms, mixtures of processed probiotic organism cell components, and mixtures of nutrients and one or more components derived from probiotic organism cells. In one embodiment, the probiotic derivative may comprise processed cell fractions which contain, for instance, metabolites or hydrolysates. In another aspect, a mixture made up of beneficial nutrients and metabolites that are produced by yeast, such as the EpiCor® brand fermentate, which is commercially available from Embria Health Sciences, LLC, may be used in accordance with the present disclosure.

Regardless of the probiotic(s) selected, the lipid multiparticulate of the present disclosure can be made very economically and can contain relatively large amounts of one or more probiotics. The composition of the present disclosure, for instance, can contain one or more probiotics, in an amount greater than about 1% by weight, such as in an amount of about 5% by weight or more, such as in an amount of about 10% by weight or more, such as in an amount of about 15% by weight or more, such as in an amount of about 20% by weight or more, such as in an amount of about 25% by weight or more, such as in an amount of about 30% by weight or more, such as an amount of about 35% by weight or more, such as an amount of about 40% by weight or more, up to about 80% by weight or less, such as in an amount of about 75% by weight or less, such as in an amount of about 70% by weight or less, based on the total weight of the lipid multiparticulate particles containing the one or more probiotics, or any ranges or values therebetween.

In accordance with the present disclosure, one or more probiotics are incorporated into a liquid matrix to form lipid multiparticulates. Examples of liquid matrices are described, for instance, in U.S. Patent Publication No. 2018/0125863, which is incorporated herein by reference. In one embodiment, the lipid matrix is different than forming micelles, microemulsions, macroemulsions, or liposomes.

The lipid matrix used to form the particles of the present disclosure, for instance, can be made from or can include many different lipid-based components, various different acid-resistant components, and the like. Examples of materials that can be used to form the liquid matrix include a fatty alcohol, a fatty acid, a fatty acid ester of a glycol and a poly glycol, a fatty acid ester of glycerol, polyglycerol, a polyglycolized glyceride, a C10-C18 triglycerides stearoyl polyoxylglyceride, a lauroyl macrogol-32 glyceride, a caprylocaproyl macrogol-8 glyceride, an oleoyl macrogol-6 glyceride, a linoleoyl macrogol-6 glyceride, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, a propylene glycol fatty acid ester, esterified alpha-tocopheryl polyethylene glycol succinate, a propylene glycol monolaurate (C12) ester, polyoxyl 35 castor oil, polyoxyl 40 hydrogenated castor oil, a lecithin, vitamin E, tocopheryl polyethylene glycol succinate (TPGS), a sugar fatty acid ester, a sorbitan fatty acid ester, a polyoxyethylene sorbitan fatty acid ester, a polyoxyethylene-polyoxypropylene copolymer, propylene glycol, triacetin, isopropyl myristate, diethylene glycol monoethyl ether, polyethylene glycol, glycerol, mixtures or combinations thereof.

However, as discussed above, according to the present disclosure, the liquid matrix is formed from at least one low flow point excipient.

For example, in certain embodiments the lipid matrix may contain one or more low-flow point excipients. Low flow point excipients generally include fatty alcohols, fatty acids, fatty acid esters of glycols and poly glycols, fatty acid esters of polyglycerol and fatty acid esters of glycerol (glycerides) with flow points of less than 50° C. When the low flow point excipient is a relatively pure material, the melting point is also less than 50° C. A preferred class of low flow point excipients are low flow point glycerides. By “low flow point” excipient, such as a glyceride, is meant that the melting point of the excipient, such as a glyceride, is less than 50° C. In some embodiments, the low flow point glyceride has a melting point of less than 40° C. In some embodiments, the low-flow point excipient, such as glyceride, is a mixture of compounds, having a flow point of 50° C. or less. In some embodiments, the low-flow point excipient, such as glyceride, has a flow point of 40° C. or less. In some embodiments, the low-flow point glyceride has a low flow point of 30° C. or less. Exemplary low flow point glycerides include polyglycolized glycerides, such as some of the Gelucire products manufactured by Gattefosse, such as Gelucire® 43/01 having a nominal melting point of 43° C. Mixtures of low flow point glycerides are also effective, such as mixtures of Gelucire® 43/01 (C10-C18 triglycerides), Gelucire® 50/13 (stearoyl polyoxylglycerides), Gelucire® 44/14 (lauroyl macrogol-32 glycerides), and mixtures thereof. Other glycerides may also be used, such as fatty acid esters of glycols and poly glycols, and fatty acid esters of polyglycerols.

In addition to the benefits discussed above, the low flow point excipient may also ensure that at least a significant portion of the formulation matrix softens when ingested orally by a patient, at the temperature of the GI tract (about 37° C. for humans). This allows the formulation to break down by digestion in the gastro-intestinal (GI) tract, and ultimately to disperse in the GI tract to promote dissolution and absorption of the active. In certain embodiments the low flow point excipient provides a significant portion of the formulation matrix to be present in a non-crystalline liquid or amorphous state when ingested and softened in the GI tract.

Exemplary low flow point fatty alcohols include myristyl alcohol (Tm 38° C.), lauryl alcohol (Tm 23° C.), and capric alcohol (Tm 7° C.) . . . . Exemplary low flow point fatty acids include lauric acid (Tm 44° C.) and oleic acid (Tm 16° C.).

In another aspect, the lipid matrix includes a mild-flow point excipient. For example, in certain aspects the lipid matrix may contain one or more mild-flow point excipients. By “mild flow point” excipient is meant an excipient that has a flow point 50° C. to about 65° C. Mild flow point excipients may also have a melting point above 50° C. Mild flow point excipients generally include fatty alcohols, fatty acids, fatty acid esters of glycols and poly glycols, fatty acid esters of polyglycerol, fatty acid esters of glycerol (glycerides), waxes, polar waxes and other materials with flow points of greater than 50° C. up to about 65° C. Mild flow point excipients generally include fatty alcohols, fatty acids, fatty acid esters of glycols and poly glycols, fatty acid esters of polyglycerol, fatty acid esters of glycerol (glycerides), waxes, polar waxes and other materials with flow points of greater than 50° C. up to 65° C. A preferred class of mild flow point excipients are “mild flow point glycerides”. By mild flow point glyceride is meant that the flow point or melting point of the glyceride is greater than 50° C. up to 65° C. In some aspects, the mild-melting point glyceride is a mixture of compounds, having a flow point of greater than 50° C. up to 65° C.

In certain aspects, the lipid matrix can also include one or more high-flow point excipient. For example, in certain aspects the lipid matrix may contain one or more high-flow point excipients. By “high flow point” excipient is meant an excipient that has a flow point of greater than 65° C. High flow point excipients may also have a melting point above 65° C. High flow point excipients generally include fatty alcohols, fatty acids, fatty acid esters of glycols and poly glycols, fatty acid esters of polyglycerol, fatty acid esters of glycerol (glycerides), waxes, polar waxes and other materials with flow points of greater than 65° C. A preferred class of high flow point excipients are “high flow point glycerides”. By high flow point glyceride is meant that the flow point or melting point of the glyceride is greater than 65° C. In some aspects, the high-melting point glyceride is a mixture of compounds, having a flow point of greater than 65° C. In some aspects, the high-flow point glyceride has a flow point of 70° C. or more.

Exemplary high and mild flow point glycerides include glycerol behenate, glycerol dibehenate, glycerol palmitate, hydrogenated castor oil, and mixtures thereof. Often, the high flow point glyceride is a mixture of compounds that are formulated into a product and sold under a variety of trade names. Exemplary high and mild flow point and high and mild melt point fatty alcohols include stearyl alcohol (Tm 58° C.) and behenyl alcohol (Tm 71° C.). Exemplary high flow point and high melt point fatty acids include palmitic acid (Tm 63° C.) and stearic acid (Tm>70° C.). Exemplary waxes include paraffin wax, beeswax, candelilla wax, carnauba wax, and mixtures thereof.

A function of the high flow point excipient is to aid in the manufacturability of the particles by enabling the particles to congeal at a lower temperature to obtain solid particles during the melt-spray-congeal processing that will be discussed in greater detail below. In certain aspects the high flow point excipient aids the physical stability of the formulation. In most embodiments, the high flow point excipient is not appreciably digested in the GI tract.

However, in one aspect, it should be understood that substantially all of the excipients and/or encapsulants used to form the lipid matrix can have a melting point according to the low-melting point excipients above. Additionally or alternatively, in one aspect, when one or more excipients and/or encapsulants are used, the one or more excipients and/or encapsulants are selected so as to maintain an average melting point less of about 70° C., or, in one aspect, about 65° C. or less.

Nonetheless, regardless of the low, mild, and high melt point excipients or encapsulants, or combinations thereof selected, the one or more excipients and/or encapsulants used to form the lipid matrix also have a low water activity. Thus, in one aspect, one or more of the excipients and/or encapsulants have a water activity of about 0.33 or less. However, in one aspect, it should be understood that substantially all of the excipients and/or encapsulants used to form the lipid matrix can have a water activity according to the above. Namely, as discussed above, by using one or more excipients and/or encapsulants in the lipid matrix that have a low water activity, stability and shelf life of the probiotics may be even further improved.

Nonetheless, in some aspects, the lipid matrix of the particles may include other excipients to improve the performance and chemical stability of the formulations so long as they do not denature or harm the viability of the probiotics (e.g., do not have high water activity, or have suitable melting points). In some aspects, a dispersing agent is included in the particles. Exemplary dispersing agents include lecithin, glycerol monostearate, ethylene glycol palmitostearate, aluminum oxide, polyethylene alky ethers, sorbitan esters, and mixtures thereof. In one embodiment, the particles include an antioxidant to maintain chemical stability of the active agent. Exemplary antioxidants include vitamin E, tocopheryl polyethylene glycol succinate (TPGS), rosemary extract, ascorbic acid, ascorbyl palmitate, butylated hydroxyanisole (BHA), butylated hydroxytoluene (BHT), and mixtures and combinations thereof.

In some aspects, a flow aid is used to improve the flow properties of the particles. Exemplary flow aids also known as glidants include silica, calcium silicate, cab-o-sil, silicon dioxide, calcium phosphate tribasic, colloidal silicon-dioxide, magnesium silicate, magnesium trisilicate, starch, talc, and other flow aids.

In one aspect, the dietary composition further contains a disintegrating agent. The disintegrating agent, for example, can be a cross-linked carboxymethyl cellulose, such as croscarmellose. Croscarmellose is a cross-linked carboxymethyl cellulose salt. In one aspect, the cross-linked carboxymethyl cellulose can be a sodium salt. In one embodiment, the cross-linked carboxymethyl cellulose can be in the form of fibers or particles. The fibers or particles can form a free-flowing powder that is typically white in color. The cross-linked carboxymethyl cellulose is hydrophilic but also insoluble. Once placed in contact with a liquid, the cross-linked carboxymethyl cellulose wicks the fluid and begins to swell. The swelling action of the cross-linked carboxymethyl cellulose causes the dietary composition to disintegrate. In this manner, the cross-linked carboxymethyl cellulose can be used to control the release of the one or more probiotics.

The ability of the disintegrating agent to affect release of the probiotic can be controlled by controlling the type of cross-linked carboxymethyl cellulose incorporated into the composition and by controlling the amount of the disintegrating agent added to the composition. For example, the ability of the cross-linked carboxymethyl cellulose to swell can depend upon the hydration of the carboxymethyl groups by controlling the degree of substitution within the cross-linked cellulose polymer. The degree of substitution, for instance, can be greater than about 0.5, such as greater than about 0.55, such as greater than about 0.6, such as greater than about 0.65, such as greater than about 0.7, such as greater than about 0.75, such as greater than about 0.8. The degree of substitution is generally less than about 0.9, such as less than about 0.85, such as less than about 0.8, such as less than about 0.75. The degree of substitution can be determined by elemental analysis.

The amount of the disintegrating agent or the cross-linked carboxymethyl cellulose incorporated into the dietary composition can generally be greater than about 0.5% by weight, such as greater than about 1% by weight, such as greater than about 3% by weight, such as greater than about 5% by weight, And generally less than about 15% by weight, such as less than about 12% by weight, such as less than about 10% by weight, such as less than about 8% by weight.

The particles described herein are solid at ambient temperature and are generally spherical in shape. By generally spherical is meant that while most particles are essentially spherical, they do not necessarily form “perfect” spheres. Such particle variations in spherical shapes are known to those persons of ordinary skill in the art of melt-spray-congeal processing and similar particulate forming methods.

The particles may have a size ranging from an average diameter greater than about 1 μm, and generally greater than about 10 μm. Typically the particles have a size ranging from an average diameter about 40 μm to about 3000 μm, such as from about 50 μm to about 2500 μm, such as from about 80 μm to about 2000 μm, such as from about 100 μm to about 1500 μm, such as from about 150 μm to about 1500 μm, such as from about 200 μm to about 1250 μm. To measure the diameters of the particulates, there are several methods that can be used, including laser diffraction, optical microscopy, and/or SEM.

In certain aspects, the particles containing the active ingredient and lipid matrix have a flow point above 25° C., such as above 30° C., such as above 35° C., such as above 40° C. up to about 65° C.

In one aspect, the lipid matrix composition can include greater than 50 wt % of the low flow point excipient. In one embodiment, the lipid matrix composition comprises at least 2 wt % of the high flow point excipient. In another embodiment, the lipid matrix composition comprises less than 20 wt % of the high flow point excipient. In another embodiment the mass ratio of the low flow excipient to the high flow excipient is at least 2:1. In still another embodiment, the mass ratio of the low flow excipient to the high flow excipient is at least 3:1. In another embodiment, the mass ratio of the low flow excipient to the high flow excipient is at least 4:1. In another embodiment, the mass ratio of the low flow excipient to the high flow excipient is at least 10:1. In another embodiment, the mass ratio of the low flow excipient to the high flow excipient is at least 15:1. In another embodiment, the mass ratio of the low flow excipient to the high flow excipient is at least 20:1.

In one particular embodiment, the lipid matrix contains a wax combined with a fatty acid alcohol and a fatty acid. The wax, for instance, can comprise candelilla wax. The fatty alcohol, on the other hand, can be stearyl alcohol, while the fatty acid can be stearic acid. For example, the wax, such as candelilla wax, can be present in the composition in an amount greater than about 1% by weight, such as in an amount greater than about 5% by weight, and generally in an amount less than about 10% by weight, such as in an amount less than about 5% by weight. The fatty alcohol, on the other hand, can generally be present in an amount greater than about 10% by weight, such as in an amount greater than about 12% by weight, and generally in an amount less than about 25% by weight, such as in an amount less than about 22% by weight, such as in an amount less than about 18% by weight. The fatty acid, on the other hand, can be present in the composition in an amount greater than about 20% by weight, such as in an amount greater than about 25% by weight, such as in an amount greater than 30% by weight, and generally in an amount less than about 50% by weight, such as in an amount less than about 30% by weight, such as in an amount less than about 20% by weight.

As noted above, the lipid matrix the particles may include other excipients so long as they do not denature or harm the viability of the probiotics (e.g., do not have high water activity, or have suitable melting points). Exemplary addition excipients include dispersing agents, antioxidants, flow aids, flavorings, and/or sweeteners.

The lipid matrix may also comprise a dispersing agent. In one embodiment, the lipid matrix is comprised of from 0 wt % to 20 wt %, such as from 0.01 wt % to 20 wt %, of a dispersing agent. In another embodiment, the lipid matrix is comprised of from 2 wt % to 10 wt % of a dispersing agent.

The lipid matrix may also comprise an antioxidant. In one embodiment, the lipid matrix comprise from 0 wt % to 20 wt %, such as from 0.01 wt % to 10 wt %, of an antioxidant. In one embodiment, the lipid matrix comprise from 1 wt % to 5 wt % of an antioxidant.

The lipid matrix may also comprise a flow aid. In one embodiment, the lipid matrix may comprise from 0 wt % to 5 wt %, such as from 0.01 wt % to 5 wt %, of a flow aid. In another embodiment, the lipid matrix may comprise from 0.5 wt % to 2 wt % of a flow aid.

The lipid matrix may also contain flavoring or sweeteners to improve the taste of the particles to the user. In one embodiment, the lipid matrix comprise from 0 wt % to 15 wt %, such as from 0.01 wt % to 10 wt %, of an flavoring or sweetener. In one embodiment, the lipid matrix comprise from 1 wt % to 5 wt % of an antioxidant flavoring or sweetener. Flavoring and sweeteners include essential oils other sweeteners used in the nutraceutical or food industries.

Nonetheless, while other suitable processes may be used that maintain the gentle temperatures (e.g. 40 to 60° C.) of the present disclosure, in one aspect, the lipid matrix may be formulated by a suitable melt-spray-congeal process.

A molten mixture is formed by mixing and heating the lipid matrix compositions as previously described. “Molten mixture” means that the mixture of an active ingredient and lipid matrix materials are sufficiently mixed and heated to fluidize the mixture sufficiently to allow it to be atomized into droplets. Generally, the mixture is molten in the sense that it will flow when subjected to one or more forces such as pressure, shear, and centrifugal force, such as that exerted by a centrifugal or spinning-disk atomizer.

Once the molten mixture has been formed, it is delivered to an atomizer that breaks the molten mixture into small droplets. Virtually any method can be used to deliver the molten mixture to the atomizer. In certain embodiments of the disclosed methods the molten mixture is delivered to the atomizer by use of pumps and/or various types of pneumatic devices such as pressurized vessels or piston pots or extruder. In certain embodiments the molten mixture is maintained at an elevated temperature during delivery to the atomizer to prevent its solidification and to keep it in a flowable state.

When a centrifugal atomizer (also known as rotary atomizers or spinning-disk atomizer) is used, the molten mixture is fed onto a rotating surface, where it spreads outward and flows by centrifugal force. The rotating surface may take several forms, examples of which include a flat disk, a cup, a vanned disk, and a slotted wheel. The surface of the disk may also be heated to aid in atomization of the molten mixture or cooled to aid in the solidification of the cores containing the lipid matrix. Several mechanisms of atomization are observed with flat-disk and cup centrifugal atomizers, depending on the flow of molten mixture to the disk, the rotation speed of the disk, the diameter of the disk, the viscosity of the feed, and the surface tension and density of the feed. At low flow rates, the molten mixture spreads out across the surface of the disk and when it reaches the edge of the disk, forms a discrete droplet, which is then flung from the disk.

Once the molten mixture has been atomized, the droplets are congealed, typically by contact with a gas at a temperature below the solidification temperature of the composition. Typically, it is desirable that the droplets are congealed in less than 60 seconds, less than 10 seconds, or even in less than 1 second. In certain embodiments congealing at ambient temperature using an ambient temperature cooling medium, results in sufficiently rapid solidification of the droplets. However, as certain aspects of the disclosed compositions include at least 50 wt % of a low flow point excipient, it is often preferred to utilize a cooling medium that is at a temperature that is at least 10° C. below ambient temperature. For some embodiments, it is preferred to utilize a cooling medium that is at least 20° C. below ambient temperature.

In one aspect, one or more surfactants can optionally be incorporated into the composition. Surfactants can be incorporated into the composition for various reasons. It was discovered that some surfactants can actually facilitate control of the delayed release function of the composition. In some embodiments, surfactants and co-surfactants may be included in the compositions. Exemplary surfactants and co-surfactants include polyethoxylated 12-hydroxysteric acid, also known as PEG15 hydroxy stearate (Kolliphor® HS-15), propylene glycol monocaprylate (C8) esters (Caproyl™ 90), esterified alpha-tocopheryl polyethylene glycol succinate (TPGS), mono, di, tricaprylic (C8) and capric acid (C10) esters of glycerol and mono and diesters of PEG400 (Labrasol®), Propylene glycol monolaurate (C12) esters (Labrafil® M1944CS), Polyoxyl 40 hydrogenated castor oil (Kolliphor® RH40), lecithins, and mixtures thereof.

In another aspect, the surfactant incorporated into the composition can be a polysorbate, a sulfate surfactant, or mixtures thereof. Sulfate surfactants include, for instance, salts of fatty acids sulfates. For example, in one embodiment, the surfactant can be sodium laureth sulfate.

The amounts of surfactants incorporated into the composition can vary widely depending upon the reason for adding the surfactant or the desired result. In general, when included in the composition, one or more surfactants can be present in an amount greater than about 1% by weight, such as in an amount greater than about 3% by weight, such as in an amount greater than about 7% by weight, such as in an amount greater than about 10% by weight, such as in an amount greater than about 15% by weight, such as in an amount greater than about 20% by weight, such as in an amount greater than about 25% by weight, such as in an amount greater than about 30% by weight. One or more surfactants are generally present in the composition in an amount less than about 50% by weight, such as in an amount less than about 40% by weight, such as in an amount less than about 30% by weight, such as in an amount less than about 20% by weight, such as in an amount less than about 10% by weight.

Further advantages of encapsulating the probiotic in the lipid multiparticulate include that the resulting lipid multiparticulates are stable and neutral tasting lipid multiparticulates. In addition, the lipid multiparticulates of probiotics can release the probiotics over a period of time once ingested. Further, the lipid multiparticulates may further increase the bioavailability of the probiotics. In addition, as noted above, it is a further benefit of the lipid multiparticulates that the probiotic can be present in products such as nutritional bars; and in sachet formats for adding in to oatmeal, cereals, ready-to-mix (RTM) type beverages, salads, and other similar food products, as well as pet food and treats, to achieve the benefits of the probiotic while maintaining improves stability, shelf-life, and viability. For instance, in one aspect, due to the stability of the lipid multiparticulate, the probiotic containing lipid multiparticulate, alone or with one or more other nutraceutical ingredients, can be contained in a package containing different resin materials such as high density polyethylene, aluminum, and desiccant-lined vials.

Nonetheless, in one aspect, the one or more lipid multiparticulate particles provided herein may be formulated into any suitable dosage formulation. For example, in certain aspects, the one or more particles provided herein may be placed into a capsule for delivery by oral ingestion. Exemplary capsules include hard gelatin capsules, soft gelatin capsules, HPMC capsules, as well as capsules made from other materials. The one or more particles may be suspended in an aqueous-based matrix or an oil-based matrix within the capsule itself. In certain aspects where the particles are suspended in an aqueous-based matrix or an oil-based matrix, the aqueous-based matrix or oil-based matrix may additionally include one or more active ingredients, such as the one or more nutraceutical ingredients noted above. In certain aspects, the one or more particles may be contained within a monolithic enteric capsule suitable for providing a modified release profile when ingested.

Capsules normally include a shell filled with one or more specific substances. The shell itself may be a soft or a hard capsule shell. Hard capsule shells are generally manufactured using dip molding processes, which can be distinguished into two alternative procedures. In the first procedure, capsules are prepared by dipping stainless-steel mold pins into a solution of polymer, optionally containing one or more gelling agents (e.g. carrageenan) and co-gelling agents (e.g. inorganic cations). The mold pins are subsequently removed, inverted, and dried to form a film on the surface. The dried capsule films are then removed from the molds, cut to the desired length, and then the telescoping fit caps and bodies are assembled together, printed, and packaged. In the second procedure, no gelling agents or co-gelling agents are used and film-forming polymer solution gelification on the molding pins is thermally induced by dipping pre-heated molding pins into the polymer solution. This second process is commonly referred to as thermogellation, or thermogelling dip molding. The aforementioned manufacturing processes involve the use of solutions of the different ingredients that are needed for the making the telescoping fit hard capsule shells.

Hard capsules may be filled with active ingredients, such as the particles described herein, via procedures known in the art. Typically, active ingredients are combined with various compatible excipients for ease of fill. The resulting fill may be a dry powder, a granulation, particles, lipid particles, a suspension, or a liquid. Additionally, stable, filled hard capsules have advantages over other dosage delivery forms such as liquids and solid tablets. Certain active ingredients may be difficult to formulate into dry granules or may be otherwise incompatible with the tableting process. Another consideration is improved patient compliance for taste-masking and ease of swallowing, i.e., capsules being preferred by consumers over tablets. For example, in some aspects, provided is a pharmaceutical composition that contains a capsule filled with the one or more particles disclosed herein. In some aspects, the one or more particles have not been enterically coated for modified release or gastric protection.

In certain other aspects, the one or more particles can be administered orally as a solid, liquid, suspension, or other suitable delivery vehicles. The composition of particles may be administered via buccal or sublingual administration. In one aspect, the one or more particles may be administered as a capsule, tablet, caplet, pill, troche, drop, lozenge, powder, granule, syrup, tea, drink, thin film, seed, paste, herb, botanical, and the like.

In a further aspect of the present disclosure as noted above, the lipid multiparticulate particles described herein can be combined with or used with other nutraceutical components to form a nutraceutical composition. The lipid multiparticulates of probiotics can be blended with other nutraceutical components which result in stable combinations of lipid multiparticulates of probiotics and other nutraceutical ingredients in both nutraceutical finished solid and liquid dosages, as well as in food and beverage applications. Exemplary nutraceuticals, in addition to or alternative to those discussed above, which can be blended with the lipid multiparticulates include the collagen, including hydrolyzed collagen or undenatured collagen, including but not limited to UC-II® product available from Lonza, probiotics, for example, but not limited to TWK10® product available from Lonza, enzymes, endogenous fatty acid amides, cetylated fatty acid esters, omega-3 fatty acids, hyaluronic acids, curcuminoids, herbal and botanical extracts, carotenoids, methylsulfonylmethane (MSM), carnitine, including but not limited to, Carnipure® available from Lonza, and antioxidants, for example, Oceanix™ available from Lonza. Other nutraceutical ingredients having anti-inflammatory benefits such as turmeric curcuminoids, eggshell membrane, green lipped mussel, omegas-3 EPA and DHA, krill oil, French maritime pine bark extract (Pycnogenol®), Scutellaria baicalensis and Acacia catechu extracts (Univestin®), ashwagandha extract, rose hip extract, tart cherry extract, astaxanthin, hops extract (Perluxan®), glucosamine, chondroitin, hyaluronic acid, salmon nasal cartilage, avocado soy unsaponifiable, methylsulfonylmethane (MSM), willow bark extract, tamarind seed extract, boswellic acid, palmitoylethanolamide (PEA), and cetyl myristoleate (CM), which may further eliciting anti-inflammation health benefits.

In the present disclosure, also provided is method for administering a probiotic to a mammal, which in one aspect can be over an extended period of time. The method includes orally administering to a mammal lipid multiparticulate particles, the lipid multiparticulate particles that include a lipid matrix and where probiotics are dispersed in the lipid matrix. In a typical dosage, the probiotic is typically administered to the mammal in an amount from about 0.5 Billion (B) Colony Forming units (CFU) to about 150 B CFU, for example, 1 B CFU to about 75 B CFU and more particularly between about 2.5 B CFU to 30 B CFU. Depending on the percentage of the probiotic compound in the lipid multiparticulate, the amount or number of lipid multiparticulate(s) is adjusted to achieve the correct dosage. In addition, as discussed above, the lipid multiparticulate may also exhibit a delayed release of the probiotic, such as to avoid release of the probiotic in the stomach of a user.

Nonetheless, certain embodiments of the present disclosure may be better understood according to the following examples, which are intended to be non-limiting and exemplary in nature.

### EXAMPLES

Methods used in the following examples include:

Flow Cytometry: Performed utilizing the CytoFlex S Flow Cytometer, Syto® 9, Propidium Iodide, and the accompanying method published by Beckman Coulter Life Sciences. (see, e.g. FLOW-857SP03 and FLOW-2747APP06 published by Beckman Coulter Life Sciences 2015 and 2017 respectively)

Plate Method: Lonza USGW-1248 Enumeration, MRS Agar: Largely similar to Chr. Hansen, Analytical Method QAm-017 or QAm-065 except that 1 mL sample size is used with a 0.1% Peptone Water diluent using 1:10 Dilutions with 11 grams of Raw materials and finished products, with incubation times from 48 to 72 hours.

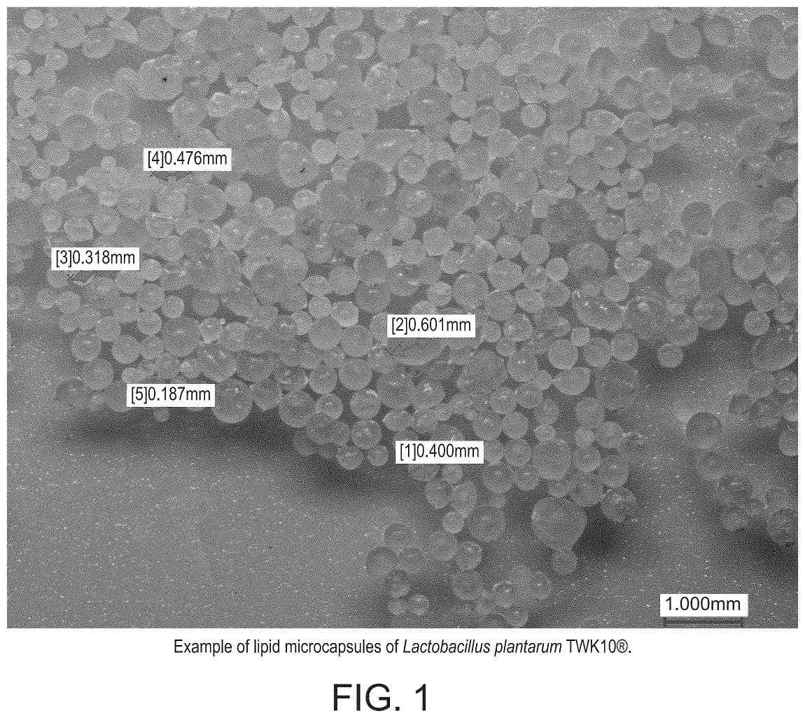

An illustration of lipid microcapsules of Lactobacillus plantarum TWK10® formed according to the present disclosure is shown in FIG. 1. For instance, lipid microcapsules were observed to range from approx. 0.2 mm to approx. 0.6 mm in size, in particular [1] 0.400 mm, [2] 0.601 mm, [3] 0.318 mm, [4] 0.476 mm, and [5] 0.187 mm in size.

### Example 1

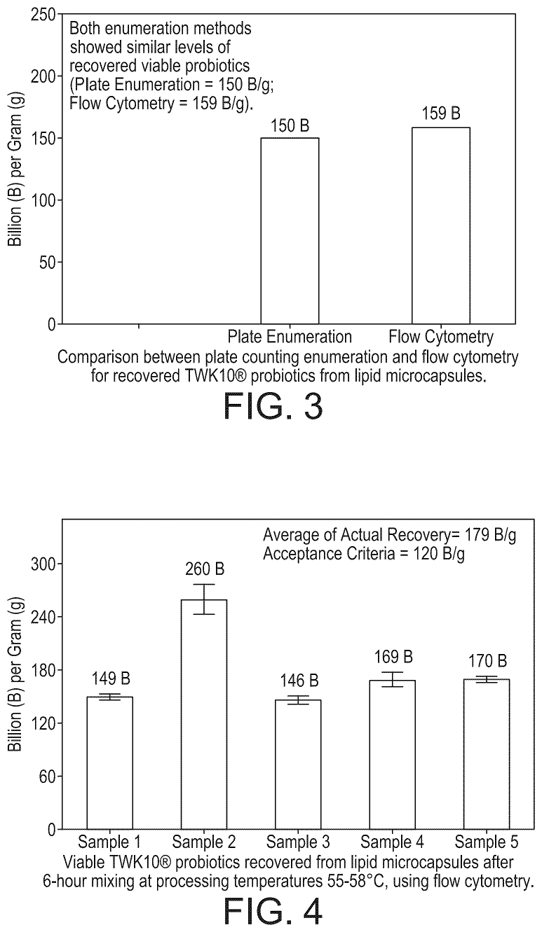

Plate enumeration method and flow cytometry method were compared, as illustrated in FIG. 3. In particular, a comparison between plate counting enumeration and flow cytometry for recovered TWK10® probiotics from lipid microcapsules was conducted. Both methods showed similar levels of recovered viable probiotics. Specifically, plate enumeration method revealed 150 B/g of recovered viable probiotics while flow cytometry method revealed 159 B/g of recovered viable probiotics. These counting methods were further utilized in the present disclosure as detailed in Example 3 below.

### Example 2

Extraction and recovery procedures were developed for probiotics from lipid microcapsules, prior to counting processes using flow cytometry, as illustrated in FIG. 2. Procedure was initiated with freeze dried probiotics (Step 1). Lipid microcapsules containing probiotics were produced according to the processes of the present disclosure (Step 2). Extractions with non-polar organic solvents were performed, in particular, (A) before mixing, (B) during mixing, (C) and (D) after mixing (Step 3). Probiotics powder was recovered after drying (Step 4).

### Example 3

A formulation was prepared from TWK10® strain containing 300 Billion/gram (B/g) viable probiotics, candelilla wax and stearic acid. Specifically, the formula contained 40% w/w TWK10®, 50% w/w stearic acid and 10% w/w candelilla wax, with a target viable probiotic content of 120 B/g. The formulations were made by mixing and agitating the ingredients, and temperature was kept between 55° C. to 65° C., until TWK10® was completely suspended in melted stearic acid and candelilla wax. The final mixture was then processed in a melt spray congeal unit, wherein lipid multiparticulates having microparticles of 300 to 900 microns were recovered. An average yield of 70% of total lipid multiparticulate solids were recovered from these processes. The actual probiotics count recovered from the formulation had an amount of 150 billion per gram (theoretical according to manufacture is 120 Billion per gram). Therefore, acceptance criteria for the examples was set at 120 Billion per gram.

From the prepared lipid multiparticulates, five individual samples were separated and tested for viable TWK10® probiotics under varying conditions, as illustrated in FIGS. 4-7. Namely, as illustrated, individually and on average, the recovered multiparticulates formed according to the present disclosure exhibited excellent viability, up to 100% viable recovery when compared to theoretical.

As shown in FIG. 4, viable TWK10@ probiotics were recovered from lipid microcapsules after 6-hour mixing at processing temperatures 55-58° C. and counted using flow cytometry. It was observed that the average of actual recovery was 179 B/g (with acceptance criteria set at 120 B/g). Specifically, 149 B/g of viable TWK10® probiotics were recovered from Sample 1, 260 B/g of viable TWK10® probiotics were recovered from Sample 2, 146 B/g of viable TWK10® probiotics were recovered from Sample 3, 169 B/g of viable TWK10® probiotics were recovered from Sample 4, and 170 B/g of viable TWK10® probiotics were recovered from Sample 5.

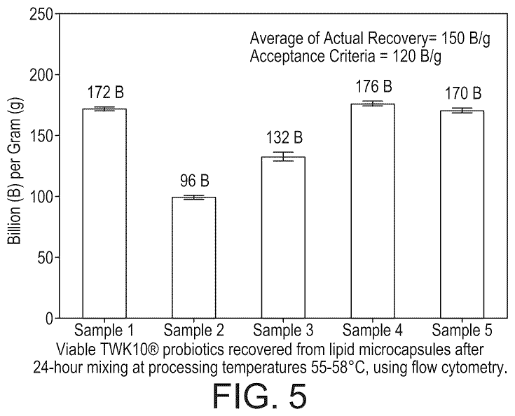

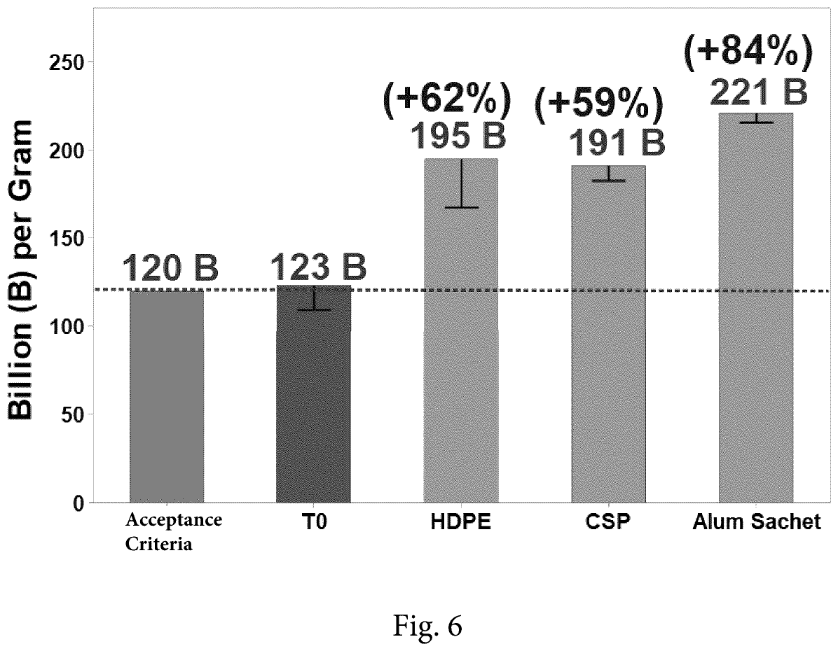

As shown in FIG. 5, viable TWK10® probiotics were recovered from lipid microcapsules after 24-hour mixing at processing temperatures 55-58° C. and counted using flow cytometry. It was observed that the average of actual recovery was 150 B/g (with acceptance criteria set at 120 B/g). Specifically, 172 B/g of viable TWK10® probiotics were recovered from Sample 1, 99 B/g of viable TWK10® probiotics were recovered from Sample 2, 132 B/g of viable TWK10® probiotics were recovered from Sample 3, 176 B/g of viable TWK10® probiotics were recovered from Sample 4, and 170 B/g of viable TWK10® probiotics were recovered from Sample 5. In addition, as shown in FIG. 6, excellent recovery was illustrated regardless of packaging. Viable TWK10® probiotics were recovered from lipid microcapsules after 6-month storage at 25° C. temperature, 60% relative humidities and counted using flow cytometry. Acceptance criteria was set at 120 B/g. At the start (TO), 123 B/g of viable TWK10® probiotics were recovered from lipid microcapsules. After storage under conditions specified above, 195 B/g of viable TWK10® probiotics were recovered from lipid microcapsules when stored in a polymeric sachet formed from high density polyethylene (HDPE), 191 B/g of viable TWK10® probiotics were recovered from lipid microcapsules when stored in a desiccant vial-CSP™ Vial, and 221 B/g of viable TWK10® probiotics were recovered from lipid microcapsules when stored in an aluminum sachet. Unexpectedly, each of the storage methods facilitated viability of the probiotics within the lipid microcapsules. For example, 62% increase in probiotic recovered from lipid microcapsules was observed when stored in HDPE under conditions noted above, 59% increase in probiotic recovered from lipid microcapsules was observed when stored in CSP™ under conditions noted above, and 84% increase in probiotic recovered from lipid microcapsules was observed when stored in aluminum sachet under conditions noted above.

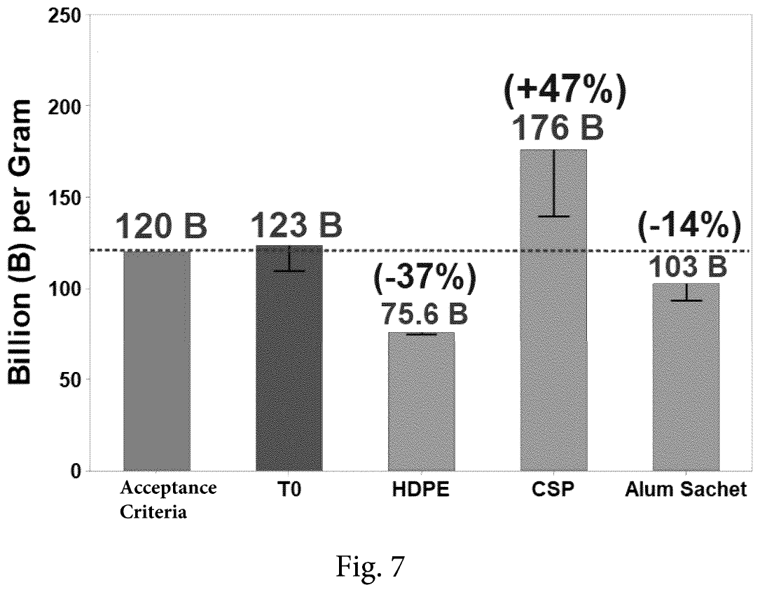

Moreover, in FIG. 7, it is shown that the lipid multiparticulates may exhibit excellent viability even when exposed to rigorous high temperature and high humidity environments. Viable TWK10® probiotics were recovered from lipid microcapsules after 6-month storage at 40° C. temperature, 75% relative humidities and counted using flow cytometry. Acceptance criteria was set at 120 B/g. At the start (TO), 123 B/g of viable TWK10® probiotics were recovered from lipid microcapsules. After storage under conditions specified above, 75.6 B/g of viable TWK10® probiotics were recovered from lipid microcapsules when stored in a polymeric sachet formed from high density polyethylene (HDPE), 176 B/g of viable TWK10® probiotics were recovered from lipid microcapsules when stored in a desiccant vial-CSP™ Vial, and 103 B/g of viable TWK10® probiotics were recovered from lipid microcapsules when stored in an aluminum sachet. Unexpectedly, each of the storage methods preserved viability of the probiotics within the lipid microcapsules. For example, 47% increase in probiotic recovered from lipid microcapsules was observed when stored in CSP™ under conditions noted above, whereas only 37% decrease in probiotic recovered from lipid microcapsules was observed when stored in HDPE under conditions noted above and only 14% decrease in probiotic recovered from lipid microcapsules was observed when stored in aluminum sachet under conditions noted above.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A probiotic composition comprising:
lipid multiparticulate particles, and
at least one probiotic and optionally a paraprobiotic,
the lipid multiparticulate particles comprising a lipid matrix, and wherein the at least one probiotic is dispersed within the lipid matrix,
wherein at least a portion of the lipid matrix is formed from one or more encapsulants, one or more excipients, or a combination thereof, having a melting point of about 70° C. or less, and
wherein greater than about 80% of the probiotics contained in the lipid matrix are viable probiotics.

2. A health composition comprising:
at least one nutraceutical ingredient, wherein the at least one nutraceutical ingredient has a water-activity of greater than 0.2, is an antimicrobial ingredient, or a combination thereof;
lipid multiparticulate particles, wherein the lipid multiparticulate particles include a lipid matrix, and wherein at least one probiotic, and optionally a paraprobiotic, is dispersed within the lipid matrix,
wherein at least a portion of the lipid matrix is formed from one or more encapsulants, one or more excipients, or a combination thereof, having a melting point of about 70° C. or less.

3. The composition as defined in claim 1, wherein the lipid matrix is formed from one or more encapsulants, one or more excipients, or a combination thereof, having an average melting point of less than about 70° C.

4. The composition as defined in claim 1, wherein at least a portion of the one or more encapsulants, one or more excipients, or a combination thereof, have a melting point of less than about 50° C.

5. The composition as defined in claim 1, further comprising at least one nutraceutical ingredient, wherein the at least one nutraceutical ingredient includes an herbal extract, a botanical extract, a coloring, a flavoring, an enzyme, a vitamin, a mineral, an omega-3 fatty acid, a prebiotic, a postbiotic, a fiber source, or a combination thereof.

6. The composition as defined in claim 1, wherein greater than about 90% of the probiotics contained in the lipid matrix are viable probiotics, wherein the percent recovery is after exposure to processing and/or a temperature of 55° C. for at least about 2 hours.

7. The composition as defined in claim 1, wherein the probiotic is present in the lipid multiparticulate particles in an amount from about 1% to about 80% by weight based on a total weight of the lipid multiparticulate particles.

8. The composition as defined in claim 1, wherein the composition is in a form of a capsule or a powder or suspended in a liquid, or wherein the composition is or is contained in, a ready-to-mix beverage, oatmeal, cereal, a nutritional bar, pet chews, pet treats, or a combination thereof, and/or wherein the composition is packaged in a resin material, wherein the resin material comprises a high density polyethylene, aluminum, or a desiccant-lined vial.

9. The composition as defined in claim 1, wherein the lipid multiparticulate particles have an average particle size of greater than 1 μm.

10. The composition as defined in claim 1, wherein the lipid matrix further comprises at least one mild flow point excipient, high flow point excipient, or a combination thereof, the at least one mild flow point excipient, high flow point excipient, or a combination thereof having an average melting point of less than about 65° C.

11. The composition as defined in claim 1, wherein the lipid matrix comprise a fatty alcohol, a fatty acid, a fatty acid ester of a glycol and a poly glycol, a fatty acid ester of glycerol, polyglycerol, a polyglycolized glyceride, a C10-C18 triglyceridesstearoyl polyoxylglyceride, a lauroyl macrogol-32 glyceride, a caprylocaproyl macrogol-8 glyceride, an oleoyl macrogol-6 glyceride, a linoleoyl macrogol-6 glyceride, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, a propylene glycol fatty acid ester, esterified alpha-tocopheryl polyethylene glycol succinate, a propylene glycol monolaurate (C12) ester, polyoxyl 35 castor oil, polyoxyl 40 hydrogenated castor oil, a lecithin, vitamin E, tocopheryl polyethylene glycol succinate (TPGS), a sugar fatty acid ester, a sorbitan fatty acid ester, a polyoxyethylene sorbitan fatty acid ester, a polyoxyethylene-polyoxypropylene copolymer, rosemary extract, propylene glycol, triacetin, isopropyl myristate, diethylene glycol monoethyl ether, polyethylene glycol, glycerol, mixtures or combinations thereof.

12. The composition as defined in claim 1, wherein the lipid matrix comprises a wax, a fatty alcohol, and a fatty acid, wherein the wax comprises candelilla wax, wherein the fatty alcohol comprises stearyl alcohol, and wherein the fatty acid comprises stearic acid.

13. The composition as defined in claim 1, wherein the lipid matrix further contains a surfactant, a cross-linked carboxymethyl cellulose salt, or combinations thereof.

14. The composition as defined in claim 13, wherein the surfactant comprises a polysorbate, a laureth sulfate, or mixtures thereof.

15. The composition as defined in claim 1, wherein at least a portion of the at least one probiotic is a freeze dried probiotic powder.

16. The composition as defined in claim 1, wherein the at least one probiotic includes Lactobacilli, Bifidobacteria, or combinations thereof.

17. The composition as defined in claim 1, further comprising one or more flow aids, antioxidant, dispersing agent and/or a flavoring or sweetener, wherein the one or more flow aids, antioxidant, dispersing agent and/or a flavoring or sweetener are not dispersed in the lipid matrix.

18. A method for administering a composition to a mammal, the method comprising:
orally administering to a mammal the composition of claim 1, each dosage administered to the mammal containing the at least one probiotic in an amount from about 0.5 Billion (B)-Colony Forming Units (CFU) to about 150 B CFU.

19. A method as defined in claim 18, wherein the composition is formulated such that the probiotic is released from the composition over a period up to about 30 hours after oral administration to a mammal.

20. A method of increasing bioavailability of at least one probiotic in a mammal, the method comprising forming a composition according to claim 1 and administering the composition to the mammal.
